(19) United States
(12) Patent Application Publication
(10) Pub. No.: US 2026/0081003 A1
(43) Pub. Date: Mar. 19, 2026

(54) SYSTEMS AND METHODS FOR IMAGE SEGMENTATION USING PROMPTABLE EMBEDDING-BASED SEGMENTATION MODELS

(71) Applicant: SUNNYBROOK RESEARCH INSTITUTE, Toronto (CA)

(72) Inventors: Chinthaka HEYN, Toronto (CA); Jonathan Eric BISHOP, Toronto (CA); Pejman Jabehdar MARALANI, Toronto (CA)

(21) Appl. No.: 19/327,408

(22) Filed: Sep. 12, 2025

Related U.S. Application Data
(60) Provisional application No. 63/696,215, filed on Sep. 18, 2024.

(51) Int. Cl. G16H 30/40; G06T 7/12; G06V 10/25

(52) U.S. Cl. CPC G16H 30/40; G06T 7/12; G06V 10/25

(57) ABSTRACT

Systems and methods are provided for performing segmentation of medical image data based on generating a spatial prompt for a promptable embedding-based segmentation model, such as, for example, an interactive vision transformer based segmentation model. At least a subset of a medical image dataset is transformed into a parameter space representation, where the dataset is processed to select a set of voxels satisfying parameter space selection criteria associated with one or more target substances (e.g. a target tissue, fluid or material). The resulting selected set of voxels is back-projected into image space, and employed to generate a region selection dataset for use as a spatial prompt for the promptable embedding-based segmentation model. The region selection dataset is provided as a spatial prompt to the promptable embedding-based segmentation model, and the promptable embedding-based segmentation model is employed to process the medical image dataset to determine a segmentation.

### CROSS-REFERENCE TO RELATED APPLICATION

This application claims the benefit of U.S. Provisional Application No. 63/696,215, titled “SYSTEMS AND METHODS FOR IMAGE SEGMENTATION USING PROMPTABLE EMBEDDING-BASED SEGMENTATION MODELS” and filed on Sep. 18, 2024, the entire contents of which are incorporated herein by reference.

### BACKGROUND

The present disclosure relates to the segmentation of medical images. More particularly, the present disclosure relates to the use of promptable embedding-based segmentation models for the segmentation of medical images.

Segmentation of medical images is crucial for various clinical and research applications. Accurate segmentation allows for precise delineation of anatomical structures, tumor boundaries, and other regions of interest, which is essential for diagnosis, treatment planning, and monitoring disease progression. Clinical applications of segmentation demand both high accuracy and speed because delayed or imprecise segmentation can lead to suboptimal treatment decisions and increased workload for healthcare providers. For example, image segmentation can be applied in the field of neuro-oncology where quantifying brain tumor size (dimensions or volume) at baseline and then at follow-up is part of routine clinical practice and important for determining response to treatment.

There are several methodologies for image segmentation, typically classified by the level of user input required. At one end of the spectrum is manual or semi-automated segmentation, such as algorithms based on thresholding, region-based, fuzzy or edge-detection which, while potentially highly accurate and requiring no training, can be labor-intensive and time-consuming.

On the other end of the spectrum are fully automated methods, often based on deep learning. These can be extremely fast and require no user intervention during deployment, but their accuracy can vary significantly depending on factors such as the quality and diversity of the training data, the specific architecture of the model, and the nature of the medical images being analyzed. Convolutional neural networks (CNNs), which employ the convolution of kernels to facilitate the recognition of patterns and features in images, have played a central role in the development of advanced deep-learning-based methods capable of segmentation of a broad range of structures and features in medical images. One notable example of a CNN-based segmentation model is the U-Net model, which was specifically designed for biomedical image segmentation. The use of deep-learning based models such as U-Net has advanced tasks such as tumor detection, organ delineation, and anomaly identification, resulting in improvements in diagnostic accuracy. Deep-learning-based segmentation models are thus becoming integral to modern medical imaging, streamlining workflows, providing decision support to radiologists, improving treatment planning, and ultimately leading to an improvement in patient outcomes.

The development of deep-learning-based models has traditionally required large datasets of segmented lesions, which is both time consuming and costly to implement. In addition, the use of trained machine learning models across different centers further requires additional training on local data representing another barrier to their implementation. Indeed, it has been found that conventional CNN-based models that are trained on medical image data can have problems in terms of performance and speed (segmentation latency). For example, fully automated state of the art segmentation models that employ a CNN-based deep learning architecture but do not include a vision transformer (e.g. U-net) can occasionally make errors in image segmentation or miss the target completely, particularly for small lesions, requiring a user to correct or redo the segmentation, which can involve substantial time delays (latency) and compromise workflow efficiency. For instance, in the case of real-time tumor margin assessment in radiation planning, a segmentation time of <2 minutes for a subcentimeter brain metastasis would be preferable to facilitate adoption from a clinical workflow perspective. A recently FDA-approved algorithm based on a 3D U-net and DeepMedic of volumetric MR imaging and CT data showed whole-brain inference times of 90 seconds and an average of 6.1 minutes/case (median 2 metastases/case) for users to finalize segmentations of brain metastases for treatment planning, which exceeds the latency that is preferable for widespread clinical adoption.

Recently, a new generation of deep-learning-based image segmentation models has emerged based on the vision transformer (ViT) architecture, which is itself based on an architecture initially developed for natural language processing. Vision Transformers (ViTs) are a type of neural network architecture that apply the transformer model, originally developed for natural language processing, to image data. Vision transformers are deep learning models that have revolutionized image processing by leveraging the self-attention mechanism to capture complex relationships across different parts of an image. This allows them to excel in various visual recognition tasks, such as image classification, object detection, and segmentation, by modeling global dependencies more effectively than convolutional neural networks (CNNs).

In vision transformers, an image is divided into a grid of fixed-size patches, similar to how text is broken into tokens in a natural language transformer model. In a typical vision transformer implementation, each patch is flattened into a one-dimensional vector and passed through a linear projection to create a patch embedding. Positional embeddings are added to these patch embeddings to retain information about the spatial positions of the patches within the image. The sequence of patch embeddings is then fed into a transformer encoder, where self-attention mechanisms encode spatial relationships among the patch embeddings.

The self-attention mechanisms of the transformer encoder allow the model to determine the relevance of each patch in relation to all other patches in the image. Specifically, self-attention involves the computation of attention weights that indicate how much influence one patch embedding should have on another when constructing a representation of the image. Since the patch embeddings are learned representations of the original patches, the relationships captured among the embeddings effectively represent the relationships among the patches themselves. This means that each patch can “attend” to every other patch through their embeddings, enabling the model to capture global relationships and long-range dependencies across the entire image. By weighing the interactions between patch embeddings, the transformer encoder effectively integrates both local details and global context, which is essential for understanding complex patterns and structures within the image.

When adapting vision transformers for image segmentation, which requires assigning a class label to each pixel, the architecture is modified to produce detailed, local (e.g. pixel-wise) predictions instead of a single classification output. In this adaptation, the classification token typically used in vision transformers for image recognition is omitted because the focus is on generating a segmentation map rather than a single class label. After the transformer encoder processes the sequence of patch embeddings, a segmentation decoder is employed. This decoder reshapes and upsamples the encoded patch representations back to the original image resolution. Techniques such as upsampling layers, per-patch classification, and refinement modules may be used to convert the coarse output of the encoder into a fine-grained segmentation map. By leveraging the ability of the transformer to model global context through relationships among patch embeddings, and therefore among the patches themselves, and incorporating a decoder that preserves spatial details, vision transformers can effectively perform image segmentation tasks, accurately assigning class labels to each pixel in the image.

As segmentation models have evolved, so has the ability of the user to provide input to guide the segmentation process. In particular, many embedding-based segmentation models, such as CNNs and vision transformers, have been adapted to facilitate the input and processing of a user prompt providing spatial information that can be used to generate embeddings that refine the segmentation process. The ability to provide user-generated spatial prompts to an embedding-based segmentation model can be beneficial in that the user is able to influence the model and potentially interact with the model. For example, the user can input specific instructions or cues, such as point, bounding box or mask prompts. These prompts guide the focus and decision-making process of the model, allowing it to tailor its output according to the guidance and preferences of the user.

Despite these improvements, the current state of the art in deep-learning-based segmentation of medical images is hampered by challenges in adapting the models to the clinical workflows and needs of radiologists and other medical professionals. While promptable embedding-based deep learning models show great promise in their ability to enable the user to influence and refine the segmentation process, the need for the user to define and customize the spatial prompts can lead to workflow complexity and unacceptable time delays in the segmentation process. Moreover, differences in user-generated spatial prompts can lead to a lack of reproducibility among different users, or the same user over time, compromising the ability to maintain diagnostic reliability and utility for comparative studies.

### SUMMARY

Systems and methods are provided for performing segmentation of medical image data based on generating a spatial prompt for a promptable embedding-based segmentation model, such as, for example, an interactive vision transformer based segmentation model. At least a subset of a medical image dataset is transformed into a parameter space representation, where the dataset is processed to select a set of voxels satisfying parameter space selection criteria associated with one or more target substances (e.g. a target tissue, fluid or material). The resulting selected set of voxels is back-projected into image space, and employed to generate a region selection dataset for use as a spatial prompt for the promptable embedding-based segmentation model. The region selection dataset is provided as a spatial prompt to the promptable embedding-based segmentation model, and the promptable embedding-based segmentation model is employed to process the medical image dataset to determine a segmentation.

Accordingly, in a first aspect, there is provided a method of performing medical image segmentation via a promptable embedding-based segmentation model, the method comprising:
processing a medical image dataset to generate a parameter space representation of the medical image dataset, the medical image dataset comprising image data associated with at least one image modality, the image data associated with each image modality characterizing a spatial dependence of a respective image parameter;
processing the parameter space representation of the medical image dataset to select a set of voxels satisfying parameter space selection criteria, wherein the parameter space selection criteria is based on known parameter space properties of a preselected substance;
processing the set of voxels identified in parameter space to back-project the set of voxels into image space, and processing the resulting back-projected image space voxels to generate a region selection dataset identifying, in image space, one or more regions of interest having voxels satisfying the parameter space selection criteria;
providing the region selection dataset as a prompt to the promptable embedding-based segmentation model, and employing the promptable embedding-based segmentation model to process the medical image dataset to determine a segmented region.

In some example implementations of the method, the promptable embedding-based segmentation model comprises a vision transformer.

In some example implementations of the method, the promptable embedding-based segmentation model comprises an interactive vision transformer-based segmentation model.

In some example implementations of the method, the promptable embedding-based segmentation model is a foundational model trained on images that include non-medical images.

In some example implementations of the method, the promptable embedding-based segmentation model comprises an image encoder and a prompt encoder, wherein the promptable embedding-based segmentation model is configured such that image embeddings and prompt embeddings are processed by a mask decoder to generate the segmented region.

In some example implementations of the method, the promptable embedding-based segmentation model is capable of generating a three-dimensional segmentation, and wherein the region selection dataset identifies one or more three-dimensional regions.

In some example implementations of the method, the promptable embedding-based segmentation model is capable of generating a two-dimensional segmentation, and wherein the region selection dataset identifies one or more two-dimensional regions within a selected image slice of the medical image dataset. The method may further comprise generating a plurality of two-dimensional segmented regions by prompting the promptable embedding-based segmentation model a plurality of times, each time employing, as a prompt, a region selection dataset associated with a different two-dimensional slice of the medical image dataset. At least two of the different two-dimensional slices may be non-parallel. The method may further comprise processing the plurality of two-dimensional segmented regions to generate a three-dimensional segmented region.

In some example implementations of the method, the region selection dataset identifies two or more non-contiguous regions within image space.

In some example implementations of the method, the medical image dataset is a multiparametric image dataset, and wherein the parameter space representation of the multiparametric image dataset is a multidimensional parameter space.

In some example implementations of the method, the medical image dataset is a monoparametric image dataset. The parameter space representation of the monoparametric image dataset may be a histogram.

In some example implementations of the method, the region selection dataset comprises one or more of a mask, a bounding box and a set of points.

In some example implementations of the method, the promptable embedding-based segmentation model and a computing system employed to process the promptable embedding-based segmentation model are selected such that a time delay associated with generation of the region selection dataset and the segmented region is less than 30 seconds.

In some example implementations of the method, the parameter space selection criteria is determined by: receiving, via a user interface displaying an image space representation of at least a subset of the medical image dataset, input from a user identifying a selected region; employing the selected region to identifying a selected set of voxels within image space; and processing a parameter space representation of the selected set of voxels to autonomously generate the parameter space selection criteria.

The method may further comprise enabling the user to dynamically view, with a latency of less than 30 seconds, an updated visualization of the segmented region based on changes made by the user to the selected region.

In some example implementations of the method, the parameter space selection criteria is determined by receiving, via a user interface displaying a parameter space representation of at least a subset of the medical image dataset, input from a user identifying a selected parameter space region; and employing the selected parameter space region to generate the parameter space selection criteria.

The method may further comprise enabling the user to dynamically view, with a latency of less than 30 seconds, an updated visualization of the segmented region based on changes made by the user to the selected parameter space region.

In some example implementations of the method, prior to generating the parameter space representation of the medical image dataset, at least one image parameter is normalized according to a z-score.

In another aspect, there is provided a system for performing medical image segmentation via a promptable embedding-based segmentation model, the system comprising:
processing circuitry comprising at least one processor and associated memory, the memory storing instructions executable by the at least one processor for performing operations comprising:
   processing a medical image dataset to generate a parameter space representation of the medical image dataset, the medical image dataset comprising image data associated with at least one image modality, the image data associated with each image modality characterizing a spatial dependence of a respective image parameter;
   processing the parameter space representation of the medical image dataset to select a set of voxels satisfying parameter space selection criteria, wherein the parameter space selection criteria is based on known parameter space properties of a preselected substance;
   processing the set of voxels identified in parameter space to back-project the set of voxels into image space, and processing the resulting back-projected image space voxels to generate a region selection dataset identifying, in image space, one or more regions of interest having voxels satisfying the parameter space selection criteria;
   providing the region selection dataset as a prompt to the promptable embedding-based segmentation model, and employing the promptable embedding-based segmentation model to process the medical image dataset to determine a segmented region.

A further understanding of the functional and advantageous aspects of the disclosure can be realized by reference to the following detailed description and drawings.

### BRIEF DESCRIPTION OF THE DRAWINGS

Embodiments are described with reference to the accompanying drawings. In the drawings, like reference numbers can indicate identical or functionally similar elements.

FIGS. 1A and 1B illustrate example implementations of the transformation of a medical image dataset from image space to parameter space, and the generation and application of parameter space selection criteria to generate a spatial prompt for a promptable embedding-based segmentation model.

FIG. 1C illustrates the generation of various example forms of spatial prompts (region selection datasets) according to the present parameter-space-based processing methods based on the processing of multiple medical image slices.

FIG. 2 is a flow chart illustrating an example method of performing medical segmentation via the use of parameter space pre-processing of a medical image dataset to generate a spatial-domain prompt for a promptable embedding-based segmentation model.

FIGS. 3A and 3B schematically illustrate non-limiting examples of promptable embedding-based segmentation models.

FIG. 4 illustrates the transformation of a monoparametric medical image dataset from image space to parameter space, and the generation and application of parameter space selection criteria to generate a spatial prompt for a promptable embedding-based segmentation model.

FIG. 5 shows an example system for performing medical segmentation via the use of parameter space pre-processing of a medical image dataset to generate a spatial-domain prompt for a promptable embedding-based segmentation model.

FIG. 6 shows axial and coronal images of a 3D connected object derived by backprojecting parameter space data using the Background LAyer STastics (BLAST) methodology for a brain metastasis (left column). The 3D connected object derived from BLAST was used to generate multiple 2D bounding boxes which acted as spatial prompts for a promptable embedding-based segmentation model (Segment Anything Model, SAM). The resulting final segmentation output for the volume is shown in the right-hand column.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. Unless otherwise specified, the terms “about”and “approximately”mean plus or minus 25 percent or less.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

As used herein, the term “on the order of”, when used in conjunction with a quantity or parameter, refers to a range spanning approximately one tenth to ten times the stated quantity or parameter.

Unless defined otherwise, all technical and scientific terms used herein are intended to have the same meaning as commonly understood to one of ordinary skill in the art. Unless otherwise indicated, such as through context, as used herein, the following terms are intended to have the following meanings:

As used herein, the phrase “promptable embedding-based segmentation model” refers to a deep learning-based architecture that combines image embeddings with spatial prompts to produce a segmentation output.

As used herein, and as applied to medical image data, the phrase “parameter space” refers to a dimensional space in which each dimension corresponds to a specific imaging parameter (e.g., signal intensity, tissue diffusion, perfusion, or other quantitative metrics) associated with each imaging modality in a mono-or multiparametric medical image dataset. The coordinates in a parameter space represent parameters values of voxels or regions within the medical image dataset.

As explained above, the current state of the art in the use of promptable embedding-based segmentation models for segmentation of medical images is hampered by the high latency and performance issues of traditional models, workflow complexity and time delays associated with the need for the user to define and customize the spatial prompts, and potentially poor reproducibility due to differences in user-generated spatial prompts.

The present inventors realized that in order for promptable embedding-based segmentation models to be clinically viable, such models would benefit from a robust methodology for generating prompts that result in consistently and reproducible final segmentations. The prompt generation methodology would need to be fast, accurate, and reproducible because timely and precise feedback can be critical in clinical settings where delayed or imprecise results can lead to suboptimal treatment decisions and increased workload for healthcare providers. A solution was therefore sought that would employ an autonomous, computer-implemented or computer-assisted method for spatial prompt generation with low latency and improved segmentation accuracy and reproducibility.

One significant problem with conventional user-based methods of spatial prompt generation arises from a lack of confidence and repeatability in the user's ability to select appropriate spatial regions in the image domain to use for generation of a spatial prompt. Indeed, the very nature of spatial variations among images makes it very difficult for a user to consistently select appropriate regions within medical images for spatial prompt generation. However, if the medical image data is first transformed into parameter space, that is, an alternative space in which each dimension corresponds to a specific imaging parameter associated with a respective imaging modality, then the resulting parameter space representation is absent of spatial information, and is instead indicative of associations between voxels based on parameter values, rather than spatial information. As will be explained below, this parameter space representation of medical image data enables the generation of a spatial prompt in a rapid and consistent manner that avoids the aforementioned drawbacks associated with spatial prompts generated by user input based on an image space representation of the medical image dataset.

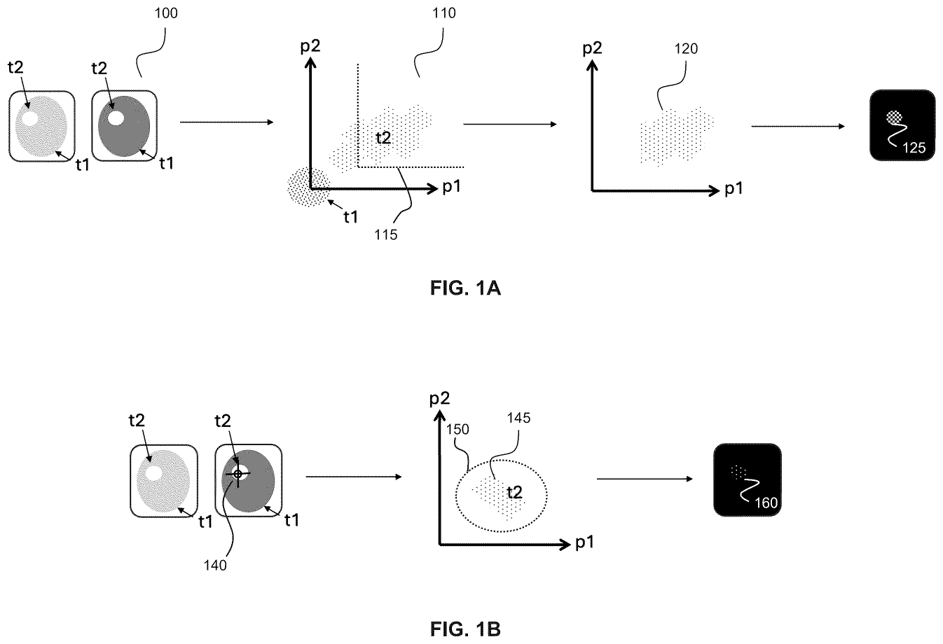

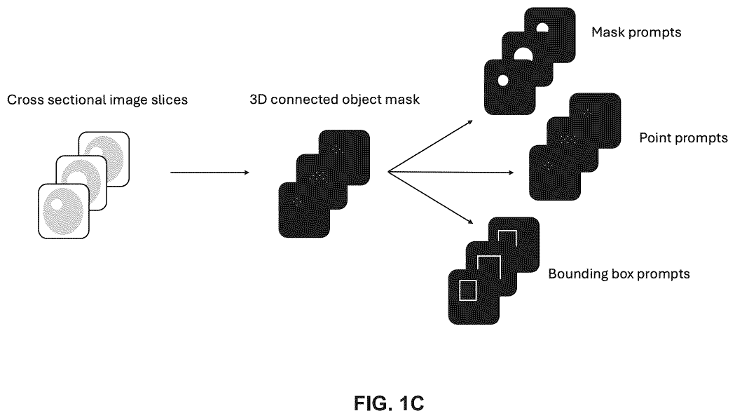

When representing a medical image dataset in a parameter space, voxels associated with different substances tend to cluster within different regions of parameter space. For example, a parameter space representation of a medical image dataset will typically show clustering among different types of substances, such as among different tissue types (e.g. normal and/or abnormal tissue types), anatomical structures, fluids, and/or non-biological materials (such as, for example, medical implants or injected media). For example, in FIG. 1A, the image space of a spatially co-registered multiparametric image dataset is illustrated at 100. Images obtained with two different image modalities are shown, with each having different regions t1 and t2 corresponding to different types of tissue (for example, tissue region t1 may correspond to normal tissue, and tissue region t2 may correspond to an abnormal tissue). When this multiparametric image dataset is transformed into parameter space, as shown at 110, the voxels from the image region corresponding to tissue type t1 cluster in a different region of parameter space than the voxels from the image region corresponding to tissue type t2.

This clustering voxels corresponding to different types of substances in parameter space enables the application of criteria (e.g. thresholds or regions) within parameter space to identify voxels corresponding to different substances, which can be performed without relying on spatial information in the original medical image dataset. For example, FIG. 1A shows an example of the application of parameter space selection criteria in the form of a parameter space threshold 115 defined such that voxels having parameters that reside above and to the right of the boundary 115 satisfy the parameter space selection criteria and are associated with tissue type t2. The application of this threshold to the parameter space representation enables the identification of a set of voxels, shown at 120, that satisfy the criteria and are deemed to be associated with the tissue type t2. While FIG. 1A shows an example in which the image parameters are normalized according to a z-score prior to generating the parameter space representation of the medical image dataset, in other example implementations, the parameter space representation can be generated according to a different normalization method, or without normalization.

The selected voxels that satisfy the parameter space selection criteria can be back-projected back into image space, thus identifying, in image space, voxels that are likely to correspond to a given selected substance associated with the parameter space selection criteria. For example, in FIG. 1A, the set of voxels 120 that satisfy the parameter space selection criteria, when back-projected into image space, result in image space voxels 125, thus generating an image-space representation of voxels associated with tissue type t2, with the segmentation of these voxels being based on the application of parameter space selection criteria in the absence of having performed a direct image-space-based segmentation.

The back-projected image-space representation of voxels satisfying the parameter space selection criteria can then be employed to generate a spatial prompt (such as a mask, bounding box, set of points, or other suitable region selection dataset or data structure) for use as a prompt for a promptable embedding-based segmentation model. Moreover, as described in further detail below, if the resulting back-projected voxels and/or the resulting region selection dataset is displayed to the user (e.g. in image space on a user interface), then the user can optionally vary the parameter space selection criteria to modify the resulting form of the spatial prompt (the region selection dataset).

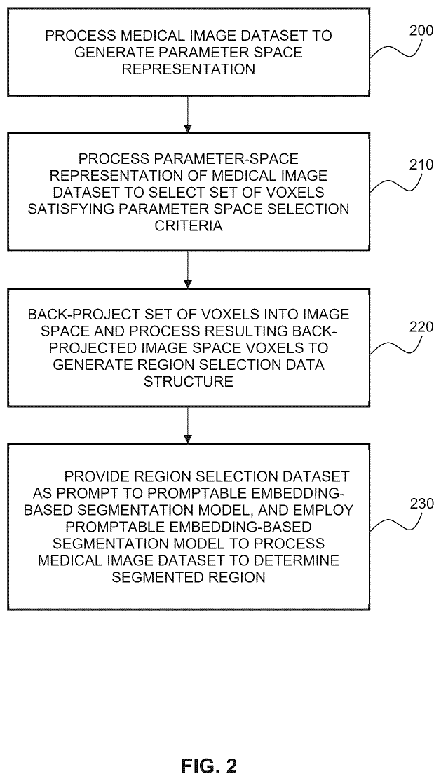

Accordingly, the systems and methods of the present disclosure provide an improved method of generating a spatial prompt (a region selection dataset) for a promptable embedding-based segmentation model, as illustrated in the flow chart shown in FIG. 2. As shown at step 200, at least a subset of a medical image dataset is processed and transformed into a parameter space representation. The parameter space representation of the medical image dataset is processed to select a set of voxels satisfying parameter space selection criteria associated with one or more target substances (e.g. a target tissue, fluid or material), as shown at step 210. The resulting selected set of voxels satisfying the parameter space selection criteria is back-projected into image space, and employed to generate a region selection dataset for use as a prompt to the promptable embedding-based segmentation model, as shown at step 220. The region selection dataset is provided as a prompt to the promptable embedding-based segmentation model, and the promptable embedding-based segmentation model is employed to process the medical image dataset to determine a segmented region, as shown at step 230.

The example method illustrated in FIG. 2 therefore facilitates the generation of a spatial prompt based on the application of selection criteria to the medical image data in parameter space instead of image space, avoiding many of the aforementioned problems associated with user-based image-space spatial prompt generation, and may be beneficial in providing low latency (fast), repeatable and scalable methods of spatial prompt generation, with low requirements for computing resources.

As illustrated above with reference to FIGS. 1A and 1B, and in FIG. 1C, the back-projected voxels that satisfy the parameter space selection criteria can be employed to generate many different forms of a region selection dataset for use as a spatial prompt to guide the segmentation of the medical image dataset by a promptable embedding-based segmentation model. In some example embodiments, the back-projected voxels may be processed to generate a mask. For example, the back-projected voxels themselves can be used directly as the spatial prompt and fed as a mask into the promptable embedding-based segmentation model. Alternatively, a subset of back-projected voxels could be used as point prompts for the model. An additional subset of pixels in close spatial proximity to the mask can be added as background pixels for a points prompt. In another example implementation, the back-projected voxels may be processed to generate a bounding box. For example, the minimum-size box enclosing all mask pixels could be selected, or a somewhat larger box with several pixels of padding around the spatial extent of the mask could be used. It will be understood that the phrase “bounding box” does not require a square or rectangular shape, and can be any closed geometrical shape that suitably encompasses the distribution of the set of back projected voxels in image space, such as, for example, a polygon and a polyhedron that might be generated with a convex hull algorithm. In other another example, at least a subset of the set of back projected voxels in image space can be employed to generate a spatial prompt based on points. It will be understood that the aforementioned example implementations involving the generation of a region selection dataset (a spatial prompt) in the form of a mask, bounding box or set of points is not intended to be limiting and that other example implementations may use alternative forms of a region selection dataset, such as, for example, a combination of bounding box and points.

Moreover, it will be understood that the region selection dataset may reside in two or three dimensions, depending on the required dimensionality of the prompt associated with the promptable embedding-based segmentation model. For example, a region selection dataset generated in the form of a mask, bounding box, and or set of points may be a 2D mask or 3D mask, a 2D or 3D bounding box, or a 2D or 3D set of points.

In some example implementations, two or more region selection datasets can be generated based on the set of back-projected voxels satisfying the parameter space selection criteria, and at least one of the resulting region selection datasets may be provided to the promptable embedding-based segmentation model. Furthermore, in some example implementations, two or more region selection datasets may be generated based on different respective parameter space thresholds, such as, for example, thresholds corresponding to different types of substances.

In some example implementations, several region selection datasets are generated that correspond to different types of substances, and at least one region selection dataset may be provided to the promptable embedding-based segmentation model as a prompt to guide the model to avoid segmentation of a selected substance (such as a selected anatomical structure or a normal/healthy type of tissue), as the promptable embedding-based segmentation model may accept both inclusive and exclusive spatial prompts, identifying regions to include or exclude during segmentation, respectively. In some example cases, a user interface may enable the user to edit, in an image space representation, the region selection dataset (the spatial prompt), optionally to identify one or more spatial regions to exclude from segmentation.

The parameter space selection criteria may be determined according to a wide variety of methods and workflows, including manual determination, semi-automated determination, and automated determination. For example, in some cases, suitable parameter space selection criteria for segmenting a particular substance (e.g. a selected tissue structure, a selected tissue type, a selected type of abnormal cells, such as a metastatic region, and a selected implant material), may be pre-determined, and autonomously applied to the medical image dataset in order to autonomously determine a set of voxels to back-project into image space for construction of the region selection dataset to be employed as a spatial prompt.

In some example embodiments, the determination of the parameter space selection criteria is based, at least in part, on input from a user. For example, in the example workflow illustrated in FIG. 1A, the parameter space representation shown at 110 may be displayed to the user on a user interface, enabling the user to select and define the parameter space selection criteria, and/or to modify an initially prescribed parameter space selection criteria, based on the display, in parameter space, of at least a portion of the medical image dataset. For example, the user may decide that a point or region should not be employed to generate the spatial prompt, and may choose to remove the point or region from the parameter space selection criteria.

In some example implementations, the parameter space selection criteria may be autonomously determined, or semi-autonomously determined, based on the selection, by a user, of a region (one or more voxels) in an image space display of at least a portion of the medical image dataset. Such an example implementation is illustrated in FIG. 1B, in which the selection, by the user, of a location or region 140 in an image space (e.g. based on the user selecting one or more points (e.g. voxels) or a collection of points) rendering of at least a portion of the medical image dataset is employed to generate a parameter space representation that includes the user-selected voxels 145 but does not include voxels within residing, in image space, beyond the user-selected voxels or region. The parameter space rendering of the user-selected voxels can then be processed to generate a suitable parameter space selection criteria.

For example, a mask defining the parameter space selection criteria may be generated within parameter space based on the statistics (e.g. distribution) of the user-selected voxels. In one example implementation, the mask may be an ellipsoid 150 with a center defined by the parameter space centroid of the user-selected voxels and diameter defined by the standard deviation of the selected voxels. To generate a suitable spatial prompt (e.g. a mask) in image space, the voxels falling within the parameter space mask defining the parameter space selection criteria are back-projected into image space.

In other example implementations, the parameter space representation may be presented to the user, and the user may apply a desired parameter space selection criteria (or modify an autonomously generated form of the parameter space selection criteria). For example, in a case in which the parameter space selection criteria is provided in the form of a parameter space mask, the parameter space mask may be edited by the user by adding or removing voxels in parameter space, or adjusting the mask location and/or size in parameter space.

While FIGS. 1A and 1B illustrate two non-limiting methods of determining and applying parameter space selection criteria, it will be understood that other methods may be employed in the alternative without departing from the intended scope of the present disclosure. For example, various example methods of generating and applying thresholds within a parameter space representation of medical image data, and the generation of image space segmentation masks, are described in International Patent Application No. PCT/CA2023/051720, titled “Systems and Methods for Detection, Segmentation and Visualization of Abnormal Regions in Medical Images”, filed on Dec. 20, 2023, which is incorporated herein by reference in its entirety.

In some example implementations, an image space representation is presented to the user on a user interface in addition to the parameter space representation, such that the user interface shows the resulting back-projected voxels that satisfy the parameter space selection criteria. In some example implementations, the effect of changes to the parameter space selection criteria on the back-projected voxels in image space may be rendered sufficiently fast to provide the user with a sense of real-time operation, such as with a latency of less than 100 ms, less than 50 ms, or less than 20 ms, or sufficiently fast to provide the user with a sense of near-real-time operation, such as with a latency less than 1 second or 500 ms. This display of the back-projected voxels may be initially rendered based on only a subset of the medical image dataset, and the application of the parameter space selection criteria to the full image dataset may be subsequently performed, for example, after receiving appropriate input from the user.

The present example embodiments may be employed to generate, based on the initial processing of a medical image dataset in parameter space according to parameter space selection criteria, a spatial prompt (a region selection dataset) for a promptable embedding-based segmentation model. It will be understood that the systems and methods disclosed herein are applicable to a broad range of promptable embedding-based segmentation models, as described in detail below.

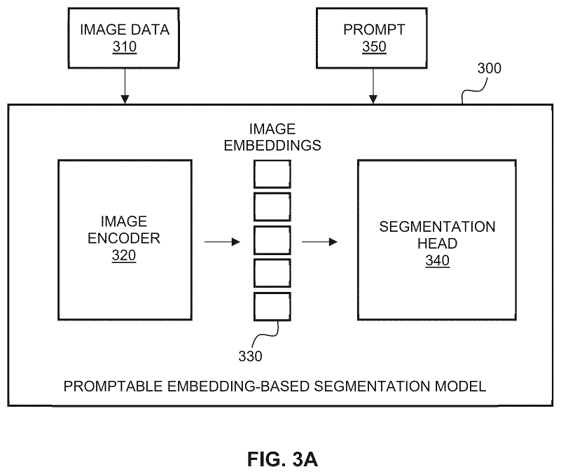

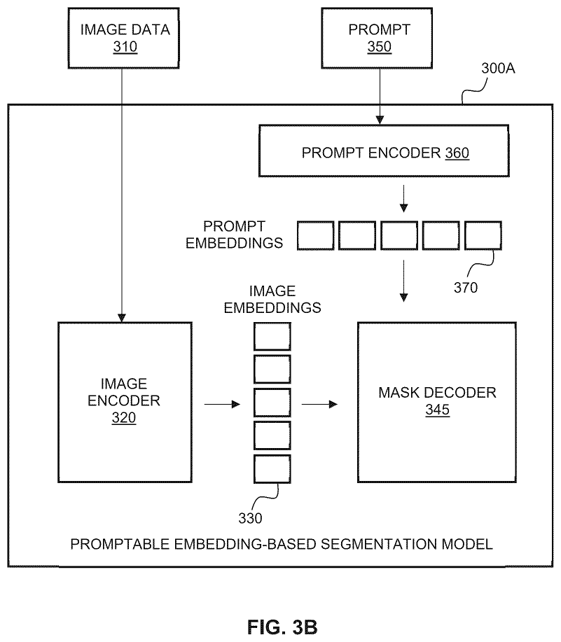

FIG. 3A schematically illustrates a promptable embedding-based segmentation model 300 that can be prompted based on a spatial prompt generated according to the parameter space processing methods disclosed herein. The example model 300 has a deep learning architecture and is structured to processes image data 310 and generate image embeddings 330 via an image encoder 320. The image embeddings 330 are processed with a segmentation head 340 (e.g. a decoding structure) to generate the final segmentation mask. The promptable embedding-based segmentation model may be, in some example implementations, a foundational type model (e.g. a general-purpose model designed to be broadly applicable across different types of images, trained on diverse datasets), a model trained specifically on medical image data, or a foundational model refined by additional training involving medical image data.

The image encoder 320 may include any suitable deep-learning-based encoder architecture for generating image embeddings, such as, but not limited to, a convolutional neural network, a recurrent neural network, a vision transformer, and hybrid architectures. The segmentation head 340 may likewise possess any suitable architecture for transforming the image embeddings into a segmentation mask (or map), and its architecture will generally be dependent on the type of image encoder 320. For example, when the image encoder 320 has a vision transformer architecture, the segmentation head 340 will be configured to process the image path embeddings generated by the transformer encoder architecture.

It is noteworthy that in implementations in which the promptable embedding-based segmentation model employs a vision transformer, the prompt generation portion of the processing pipeline may be implemented using parameter-space-based processing that is absent of spatial information (e.g. in the case of the fully autonomous processing of parameter space), while the segmentation performed by the vision-transformer-based promptable embedding-based segmentation model leverages spatial relationships among image patches to generate the resulting segmentation, resulting in an unconventional processing pipeline that combines spatially-agnostic spatial prompt generation with spatially-aware image encoding and mask decoding.

FIG. 3A also shows the incorporation of the prompt 350 into the promptable embedding-based segmentation model. This spatial prompt includes spatial information (e.g. a region selection dataset as described above) and may also optionally include text information. The spatial prompt, generated, at least in part, according to the parameter space processing methods disclosed herein, may be fused into architecture according to a wide variety of implementations, including early fusion approaches, for example, in which the spatial prompt is employed to generate prompt embeddings that are combined with the image embeddings early in the processing pipeline, or, for example, intermediate or late fusion approaches, in which, for example, image embeddings are combined with prompt embeddings in the segmentation head (e.g. a mask decoder).

On example fusion approach to spatial prompt integration is illustrated in FIG. 3B, which schematically illustrates a promptable embedding-based segmentation model 300A that includes a prompt encoder 360 for generating prompt embeddings 370, which are processed with image embeddings 330 (e.g. patch embeddings generated by the encoder of a vision transformer) in a mask decoder 345.

Some promptable embedding-based segmentation models are classified as interactive, in that the architecture of the model facilitates user interaction, such as, for example, the ability of a user to view, on a user interface, the impact of changes to the spatial prompt on the final segmentation, enabling the user to guide or influence the model's processing in real-time or near real-time. Such models typically have low inference latency, for example, less than 5 seconds, and in some cases, less than one second (e.g. depending on the model size and the computing resources available for inference). This interactivity is particularly valuable in medical imaging applications such as dynamic segmentation, where clinicians can iteratively refine the model's output to achieve more precise delineations of tumors, lesions, or other anatomical structures.

For example, promptable vision transformer models having an architecture similar to that shown in FIG. 3B can be classified as interactive, especially as the fusion of the prompt embeddings with the image embeddings enables the segmentation mask to be efficiently recomputed when changes to the spatial prompt are made by the user. An example of an interactive vision transformer based segmentation model is the Segment Anything Model (SAM) developed by Meta, which requires minimal user input in the form of prompts to guide the segmentation process. The advantage of SAM is its potential for fast, near-zero-shot operation, and with fine-tuning, it can approach the accuracy of state-of-the-art models.

Referring again to step 230 of FIG. 2, the spatial prompts generated by the application of parameter space selection criteria to the medical image dataset provide an improved and streamlined approach to spatial prompt generation for a promptable embedding-based segmentation model, such as those described above. Rather than relying on a user to provide a spatial prompt based on image annotations made in the image domain, which can be problematic due to the many reasons described above, the present parameter-space-based methods enable the rapid, efficient and repeatable generation of spatial prompts that can guide the segmentation process of a promptable embedding-based segmentation model such as an interactive vision transformer model, thereby retaining all of the benefits of such segmentation models, in terms of low inference latency and dynamic user control, while also improving the segmentation accuracy via the spatial prompt.

The segmentation generated from the promptable embedding-based segmentation model may be employed to provide a treatment to a subject, such as a radiation therapy or surgical treatment. For example, the segmentation may be employed to generate or refine a radiation or surgical treatment plan. Moreover, the segmentation may be employed to guide the choice of a particular treatment, and the timing of the delivery of the treatment.

In other example implementations, the segmentation generated by the promptable embedding-based segmentation model may be employed for classification purposes. For example, the segmentation may identify a region in the medical image dataset, such as, for example, a lesion (or set lesions), and the segmentation may be employed as a prompt (or at least a portion of a prompt) provided to a promptable embedding-based model (such as a transformer-based model) capable of providing a differential diagnosis based on the segmentation (e.g. the one or more lesions) and the medical image dataset.

When the present parameter-space-based processing methods are employed to rapidly, repeatably and efficiently generate a spatial prompt for a promptable embedding-based segmentation model that is interactive (i.e. permits the user to refine the input to the model and dynamically view the impact of the changes to the resulting segmentation), the user is able to dynamically and interactively define and refine the parameter space selection criteria that is employed to select voxels for generating the spatial prompt. Accordingly, the low-latency methods disclosed herein preserve the ability of the overall system to provide an interactive segmentation framework for the user.

For example, depending on the complexity of the model and the computing resources available, the process of generation of the spatial prompt and subsequent segmentation inference by the embedding-based segmentation model prompted with the spatial prompt may occur in less than 30 seconds, or in some cases, less than 10 seconds, or less than 5 seconds, based on computer resources and models presently available. It is expected that this latency will improve further in the future as computer hardware and model architecture continue to evolve.

Some promptable embedding-based segmentation models may be capable of generating 3D segmentations, and in such cases, the region selection datasets forming the spatial prompt may include 3D data structures, such as, for example, 3D masks, 3D bounding boxes, and 3D point collections. Other promptable embedding-based segmentation models may be capable of generating 2D segmentations, and in such cases, the region selection datasets forming the spatial prompt may include 2D data structures, such as, for example, 2D masks, 2D bounding boxes, and 2D point collections.

In the case of a promptable embedding-based segmentation model configured to generate a 2D segmentation, a final 3D segmentation can be generated by executing the promptable embedding-based segmentation model multiple times, each time based on different 2D slice with a respective 2D region selection dataset as a prompt. The resulting 2D segmentations generated by the promptable embedding-based segmentation model can then be processed to generate a final 3D segmentation, for example, by adding the multiple 2D segmentations together, and/or by the use of interpolation among 2D segmentations to generate a 3D segmentation. Such a workflow is illustrated in FIG. 1C.

Some 2D promptable embedding-based segmentation models, such as the 2D “segment anything model” (2D SAM), are known to have a problem associated with the ends of the volume. For example, if axial slices are provided as input to the model, the superior and inferior slices are difficult to segment with 2D SAM. In one example implementation, this problem could be avoided or mitigated by employing the model to perform 2D segments based on various non-parallel image slices, such as for example, multiple orthogonal image slices (e.g. multiple axial, coronal and/or sagittal image slices) and to interpolate the resulting 2D segmentations over the volume. This solution can be beneficial in enabling the use of 2D promptable embedding-based segmentation models to generate improved 3D segmentations without having to employ a 3D promptable embedding-based segmentation model.

It is to be understood that the region selection dataset (the spatial prompt) need not identify a single region in image space, but may identify two or more non-contiguous regions within image space. For example, 2D or 3D region selection datasets that are employed as spatial prompts could be contiguous voxels or could be objects that are not contiguous, either on a slice or within a 3D volume. For example, a spatial prompt may spatially identify multiple enhancing lesions within a brain. Accordingly, the spatial prompts (region selection datasets) generated according to the present disclosure can involve multiple unconnected spatial regions, for example, with spatial prompts that are not limited in space to a 2D slice, and can instead have other data structure formats such as 3D connected or disconnected spatial prompts, which can provide efficiency and time savings by avoiding the user having to specify multiple prompts.

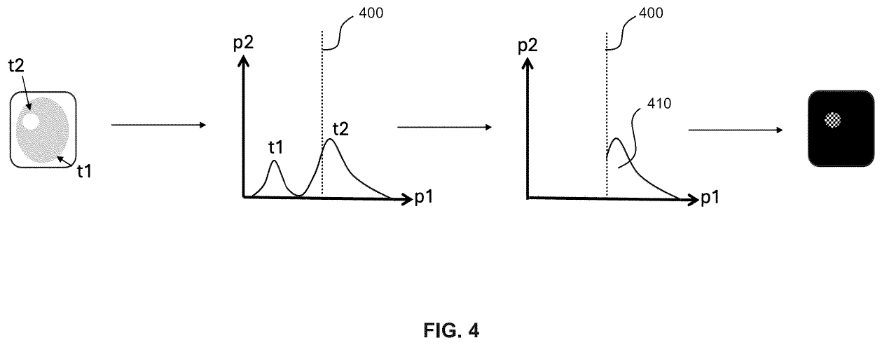

While many of the example embodiments described above have been illustrated based on the processing and segmentation of a multiparametric image dataset, the embodiments disclosed herein may be adapted for use with a monoparametric image dataset. For example, as shown in FIG. 4, a monoparametric dataset may be projected into the form of a histogram, where a parameter space threshold 400 can be applied to select a set of voxels 410 for generation of the spatial prompt. It will be understood that a histogram is intended to show one example of a parameter space representation of a monoparametric image dataset, and that other parameter space representations may be employed in the alternative, such as, for example, a density plot.

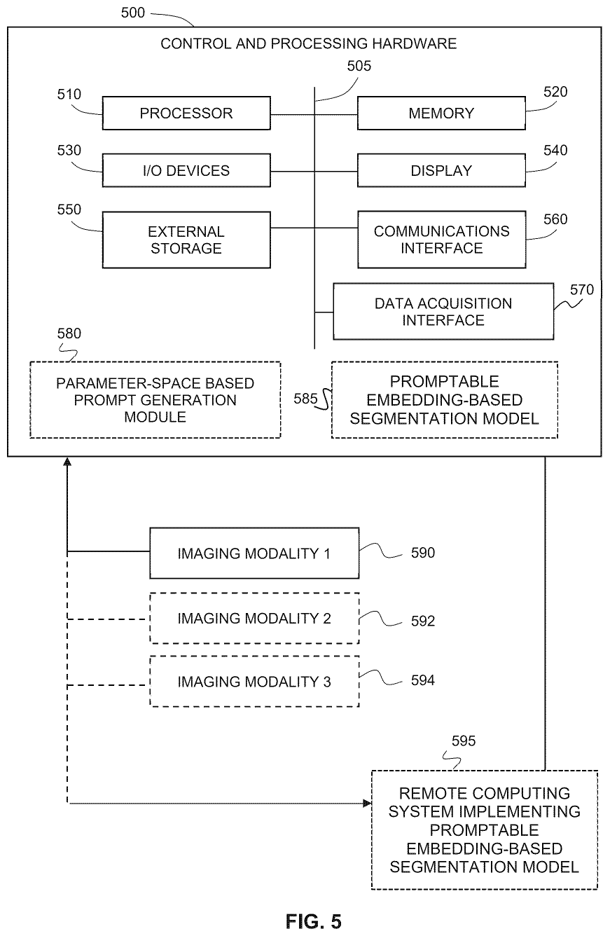

Referring now to FIG. 5, an example system is illustrated for processing medical image data to generate segmentations. The example system includes control and processing circuitry 500 which is capable of processing imaging datasets obtained from one or more imaging modality subsystems 590, 592 and 594 (non-limiting examples of which include an MRI system, a PET system, an ultrasound imaging system, and a CT system). In some example embodiments, control and processing hardware 500 may be operably coupled to one or more of the imaging modality subsystems 590, 592 and 594 (or additional imaging modality subsystems) to control acquisition of imaging datasets. In example implementations in which multiparametric imaging datasets are obtained from a common imaging modality, the imaging datasets may be obtained from a single imaging modality subsystem.

As shown in FIG. 5, in one embodiment, control and processing hardware 500 may include a processor 510, a memory 520, a system bus 505, one or more input/output devices 530, and a plurality of optional additional devices such as communications interface 560, display 540, external storage 550, and data acquisition interface 570.

The present example methods can be implemented via processor 510 and/or memory 520. As shown in FIG. 5, the example methods described above, or variations thereof, may be implemented by control and processing hardware 500, via executable instructions represented as parameter-space-based prompt generation module 580 and promptable embedding-based segmentation module 585.

The functionalities described herein can be partially implemented via hardware logic in processor 510 and partially using the instructions stored in memory 520. Some embodiments may be implemented using processor 510 without additional instructions stored in memory 520. Some embodiments are implemented using the instructions stored in memory 520 for execution by one or more general purpose microprocessors. In some example embodiments, customized processors, such as graphics processors, application specific integrated circuits (ASIC) or field programmable gate array (FPGA), may be employed. Thus, the disclosure is not limited to a specific configuration of hardware and/or software.

Referring again to FIG. 5, it is to be understood that the example system shown in the figure is not intended to be limited to the components that may be employed in a given implementation. For example, the system may include one or more additional processors. Furthermore, one or more components of control and processing hardware 500 may be provided as an external component that is interfaced to a processing device. For example, the processing of the medical image data and the spatial prompts may be performed remotely via remote computing system, as shown at 595. Alternatively, any one or more of modules 580 and 585 may be performed via one or more remote computing systems or subsystems.

While some embodiments can be implemented in fully functioning computers and computer systems, various embodiments are capable of being distributed as a computing product in a variety of forms and are capable of being applied regardless of the particular type of machine or computer readable media used to actually effect the distribution.

At least some aspects disclosed herein can be embodied, at least in part, in software. That is, the techniques may be carried out in a computer system or other data processing system in response to its processor, such as a microprocessor, executing sequences of instructions contained in a memory, such as ROM, volatile RAM, non-volatile memory, cache or a remote storage device.

A computer readable storage medium can be used to store software and data which when executed by a data processing system causes the system to perform various methods. The executable software and data may be stored in various places including for example ROM, volatile RAM, nonvolatile memory and/or cache. Portions of this software and/or data may be stored in any one of these storage devices. As used herein, the phrases “computer readable material” and “computer readable storage medium” refers to all computer-readable media, except for a transitory propagating signal per se.

### EXAMPLES

The following examples are presented to enable those skilled in the art to understand and to practice embodiments of the present disclosure. They should not be considered as a limitation on the scope of the disclosure, but merely as being illustrative and representative thereof.

### Example 1

### Segmentation of Brain Metastases

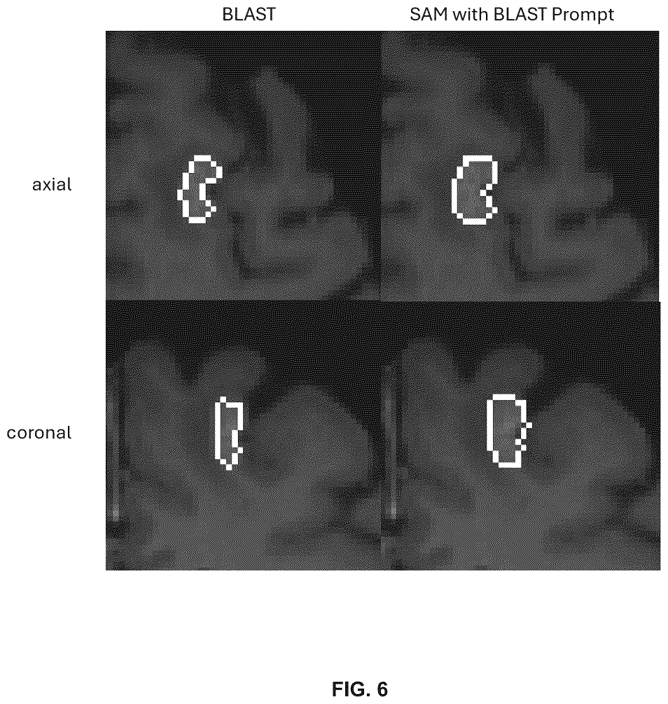

The present example involves brain metastasis segmentation which can be used to measure tumor volumes (a parameter important in assessing response to treatment) or used to derive contours for radiation planning. FIG. 6 shows axial and coronal images of a 3D connected object derived by back-projecting parameter space data using the Background LAyer STastics (BLAST) methodology for a brain metastasis (left column). The 3D connected object derived from BLAST was used to generate multiple 2D bounding boxes which acted as spatial prompts for a promptable embedding-based segmentation model (Segment Anything Model, SAM). The resulting final segmentation output for the volume is shown in the right-hand column. The final segmentation is improved over the initial back-projected mask and more completely covers the brain metastasis which will provide a more accurate measure of tumor volume and a more precise delineation of the tumor boundary for radiation planning.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

1. A method of performing medical image segmentation via a promptable embedding-based segmentation model, the method comprising:
processing a medical image dataset to generate a parameter space representation of the medical image dataset, the medical image dataset comprising image data associated with at least one image modality, the image data associated with each image modality characterizing a spatial dependence of a respective image parameter;
processing the parameter space representation of the medical image dataset to select a set of voxels satisfying parameter space selection criteria, wherein the parameter space selection criteria is based on known parameter space properties of a preselected substance;
processing the set of voxels identified in parameter space to back-project the set of voxels into image space, and processing the resulting back-projected image space voxels to generate a region selection dataset identifying, in image space, one or more regions of interest having voxels satisfying the parameter space selection criteria;
providing the region selection dataset as a prompt to the promptable embedding-based segmentation model, and employing the promptable embedding-based segmentation model to process the medical image dataset to determine a segmented region.

2. The method according to claim 1 wherein the promptable embedding-based segmentation model comprises a vision transformer.

3. The method according to claim 1 wherein the promptable embedding-based segmentation model comprises an interactive vision transformer-based segmentation model.

4. The method according to claim 1 wherein the promptable embedding-based segmentation model is a foundational model trained on images that include non-medical images.

5. The method according to claim 1 wherein the promptable embedding-based segmentation model comprises an image encoder and a prompt encoder, wherein the promptable embedding-based segmentation model is configured such that image embeddings and prompt embeddings are processed by a mask decoder to generate the segmented region.

6. The method according to claim 1 wherein the promptable embedding-based segmentation model is capable of generating a three-dimensional segmentation, and wherein the region selection dataset identifies one or more three-dimensional regions.

7. The method according to claim 1 wherein the promptable embedding-based segmentation model is capable of generating a two-dimensional segmentation, and wherein the region selection dataset identifies one or more two-dimensional regions within a selected image slice of the medical image dataset.

8. The method according to claim 7 further comprising generating a plurality of two-dimensional segmented regions by prompting the promptable embedding-based segmentation model a plurality of times, each time employing, as a prompt, a region selection dataset associated with a different two-dimensional slice of the medical image dataset.

9. The method according to claim 8 wherein at least two of the different two-dimensional slices are non-parallel.

10. The method according to claim 8 further comprising processing the plurality of two-dimensional segmented regions to generate a three-dimensional segmented region.

11. The method according to claim 1 wherein the region selection dataset identifies two or more non-contiguous regions within image space.

12. The method according to claim 1 wherein the medical image dataset is a multiparametric image dataset, and wherein the parameter space representation of the multiparametric image dataset is a multidimensional parameter space.

13. The method according to claim 1 wherein the medical image dataset is a monoparametric image dataset.

14. The method according to claim 13 wherein the parameter space representation of the monoparametric image dataset is a histogram.

15. The method according to claim 1 wherein the region selection dataset comprises one or more of a mask, a bounding box and a set of points.

16. The method according to claim 1 wherein the promptable embedding-based segmentation model and a computing system employed to process the promptable embedding-based segmentation model are selected such that a time delay associated with generation of the region selection dataset and the segmented region is less than 30 seconds.

17. The method according to claim 1 wherein the parameter space selection criteria is determined by:
receiving, via a user interface displaying an image space representation of at least a subset of the medical image dataset, input from a user identifying a selected region;
employing the selected region to identifying a selected set of voxels within image space; and
processing a parameter space representation of the selected set of voxels to autonomously generate the parameter space selection criteria.

18. The method according to claim 17 further comprising enabling the user to dynamically view, with a latency of less than 30 seconds, an updated visualization of the segmented region based on changes made by the user to the selected region.

19. The method according to claim 1 wherein the parameter space selection criteria is determined by:
receiving, via a user interface displaying a parameter space representation of at least a subset of the medical image dataset, input from a user identifying a selected parameter space region; and
employing the selected parameter space region to generate the parameter space selection criteria.

20. The method according to claim 19 further comprising enabling the user to dynamically view, with a latency of less than 30 seconds, an updated visualization of the segmented region based on changes made by the user to the selected parameter space region.

21. The method according to claim 1 wherein prior to generating the parameter space representation of the medical image dataset, at least one image parameter is normalized according to a z-score.

22. A system for performing medical image segmentation via a promptable embedding-based segmentation model, the system comprising:
processing circuitry comprising at least one processor and associated memory, the memory storing instructions executable by said at least one processor for performing operations comprising:
processing a medical image dataset to generate a parameter space representation of the medical image dataset, the medical image dataset comprising image data associated with at least one image modality, the image data associated with each image modality characterizing a spatial dependence of a respective image parameter;
processing the parameter space representation of the medical image dataset to select a set of voxels satisfying parameter space selection criteria, wherein the parameter space selection criteria is based on known parameter space properties of a preselected substance;
processing the set of voxels identified in parameter space to back-project the set of voxels into image space, and processing the resulting back-projected image space voxels to generate a region selection dataset identifying, in image space, one or more regions of interest having voxels satisfying the parameter space selection criteria;
providing the region selection dataset as a prompt to the promptable embedding-based segmentation model, and employing the promptable embedding-based segmentation model to process the medical image dataset to determine a segmented region.
